(19) United States
(12) Patent Application Publication
(10) Pub. No.: US 2025/0334586 A1
(43) Pub. Date: Oct. 30, 2025

(54) Methods, Kits and Compositions for Assessing and Treating Interstitial Cystitis

(71) Applicant: Hill's Pet Nutrition, Inc., Topeka, KS (US)

(72) Inventors: Dayakar BADRI, Lawrence, KS (US); Renea CREECH, Meriden, KS (US); Kiran PANICKAR, Lawrence, KS (US)

(73) Assignee: Hill's Pet Nutrition, Inc., Topeka, KS (US)

(21) Appl. No.: 18/268,896

(22) PCT Filed: Dec. 19, 2021
(86) PCT No.: PCT/US2021/064252

§ 371 Date: Jun. 21, 2023

Related U.S. Application Data
(60) Provisional application No. 63/129,088, filed on Dec. 22, 2020.

(51) Int. Cl. G01N 33/68; A23K 10/30; A23K 50/40

(52) U.S. Cl. CPC G01N 33/6863; A23K 10/30; A23K 50/40; G01N 33/6869; G01N 2333/5434; G01N 2800/06; G01N 2800/50; G01N 2800/52

(57) ABSTRACT

Described herein are methods and kits for—inter alia—identifying a companion animal having an increased risk of developing interstitial cystitis; along with compositions for treating same.

### CROSS-REFERENCE TO RELATED APPLICATIONS

This application is a U.S. National Stage application of PCT/US2021/064252, filed Dec. 19, 2021, which claims the benefit of priority from U.S. Provisional Application No. 63/129,088, filed Dec. 22, 2020, the contents of which are hereby incorporated herein by reference in their entireties.

### BACKGROUND

Feline interstitial cystitis (FIC), alternatively known as bladder pain syndrome (BPS), is a term used to indicate a general condition characterized by bladder pain, urinary frequency, lower urinary tract obstruction (LUTD), and nocturia in cats. FIC can be classified into two forms: an acute form which resolves spontaneously in 3 to 7 days regardless of treatment, and a chronic form which persists or recurs frequently for weeks or months. FIC is also seen in all ages and felines between the ages of 2 and 7 years are at higher risk. While various scientists have proposed possible pathomechanisms that may lead to FIC, the exact causation of FIC is not clear.

Clinical diagnosis of FIC typically involves urinalysis, urine culture and abdominal ultrasound examination. However, the diagnosis is circumstantial. Typically, FIC is diagnosed by “ruling out” other possible causes including stone formation, infection, tumor formation or renal disease. Currently, there are no consistent or specific diagnostic molecular marker(s) which may serve as reliable marker for assessing FIC. Further, all clinical studies rely heavily upon subjective pet owner observation, interpretation and quantification of clinical signs that may confirm FIC. This produces unreliable results due to the inconsistent ability of an owner to reliably quantify urination patterns and behaviors.

While several potential biomarkers have been identified and evaluated in humans, few have been evaluated in cats with FIC. Thus, there is a need in the art for a non-invasive biomarker in urine to diagnose FIC and a food formulation that alleviate the condition by improving diagnostic biomarker levels and clinical symptoms. Certain embodiments of the present invention are designed to meet these, and other ends.

### BRIEF SUMMARY

Some embodiments of the present invention provide a method for diagnosing or identifying the propensity of felines to develop interstitial cystitis. In certain embodiments, the diagnostic method comprises analyzing cytokine biomarkers. Biomarker levels may be analyzed from any bodily fluid and in the preferred embodiment the biomarker level is obtained from urine. Further, preferred biomarkers tested include but are not limited to Fms-like tyrosine kinase 3 ligand (Flt3-L) and cytokine stem cell factor (SCF).

Certain embodiments of the present invention are directed towards treating interstitial cystitis in felines comprising administering a pet food composition comprising high levels of antioxidants.

Further embodiments of the present invention provide kits for diagnosing or identifying the propensity of felines to develop interstitial cystitis.

In accordance with at least one embodiment, a method is provided for identifying a companion animal having an increased risk of developing interstitial cystitis (IC). The method typically includes analyzing a biological sample obtained from a companion animal to determine the concentration of a urinary cytokine; analyzing a biological sample from a healthy companion animal to determine the concentration of the urinary cytokine; comparing the urinary cytokine concentration in the companion animal to the urinary cytokine concentration of the healthy companion animal, wherein when the biological sample obtained from the companion animal contains a concentration of a urinary cytokine that is less than the concentration of the urinary cytokine in the healthy companion animal, then the companion animal has an increased risk of developing interstitial cystitis.

According to another embodiment, provided is a method of identifying a companion animal that would benefit from a treatment that reduces the risk of developing interstitial cystitis, wherein the treatment that reduces risk of developing interstitial cystitis comprises administering to the companion animal a composition that comprises an effective amount of fiber-bound polyphenol component. The method of identifying companion animal that would benefit from a treatment that reduces the risk of developing interstitial cystitis comprising analyzing a biological sample obtained from a companion animal to determine the concentration of a urinary cytokine; analyzing a biological sample from a healthy companion animal to determine the concentration of the urinary cytokine; comparing the urinary cytokine concentration in the companion animal to the urinary cytokine concentration of the healthy companion animal; and wherein the companion animal's urinary cytokine concentration being less than the healthy companion animal's urinary cytokine concentration, indicates that the companion animal would benefit from a treatment that reduces risk of developing interstitial cystitis.

In accordance with yet another embodiment, a method of reducing risk of developing interstitial cystitis in a feline subject comprising the steps of: analyzing a biological sample obtained from a companion animal to determine the concentration of a urinary cytokine; analyzing a biological sample from a healthy companion animal to determine the concentration of the urinary cytokine; comparing the urinary cytokine concentration in the companion animal to the urinary cytokine concentration of the healthy companion animal, wherein the companion animal's urinary cytokine concentration being less than the healthy companion animal's urinary cytokine concentration, indicates that the companion animal would benefit from treatment; and administering to the companion animal a composition comprising an effective amount of a fiber-bound polyphenol component when the companion animal's urinary cytokine concentration being less than the healthy companion animal's urinary cytokine concentration.

According to additional embodiments, provided is a kit for identifying a companion animal having an increased risk for developing interstitial cystitis. The kit typically comprises a vessel for collecting a biological sample; a detection method selected from: ELISA, chromatographic analysis, aptamer-based quantitative assay, fluorescence tags or stains specific to the combination of two or more metabolites thereof; point of care testing devices with preloaded chromophores/antibodies specific to one or more of the urinary cytokines described herein; and instructions for use.

In accordance with certain embodiments, a method is provided for treating, inhibiting or ameliorating a symptom associated with interstitial cystitis in a companion animal, comprising administering a composition comprising an effective amount of a fiber-bound polyphenol to a companion animal in need thereof.

### BRIEF DESCRIPTION OF THE DRAWINGS

FIG. 1A depicts levels of a certain urinary cytokine in healthy and diseased felines.

FIG. 1B depicts levels of a urinary cytokine before and after an interstitial cystitis diagnosis.

FIG. 1C depicts levels of a urinary cytokine over a period of years.

FIGS. 2A and 2B depict the relationship between various urinary cytokines in healthy and diseased felines.

FIGS. 3A and 3B depict the levels of two urinary cytokines in healthy and diseased felines.

FIG. 4 depicts levels of a serum cytokine in diseased and healthy felines.

FIG. 5A depicts ROC curves for diseased and healthy felines generated from urinary cytokines.

FIG. 5B depicts ROC curves for diseased and healthy felines generated from blood CBC, chemistry and cytokines.

FIG. 6A and FIG. 6B depict data related to urinary cytokines; and blood CBC, chemistry and cytokines.

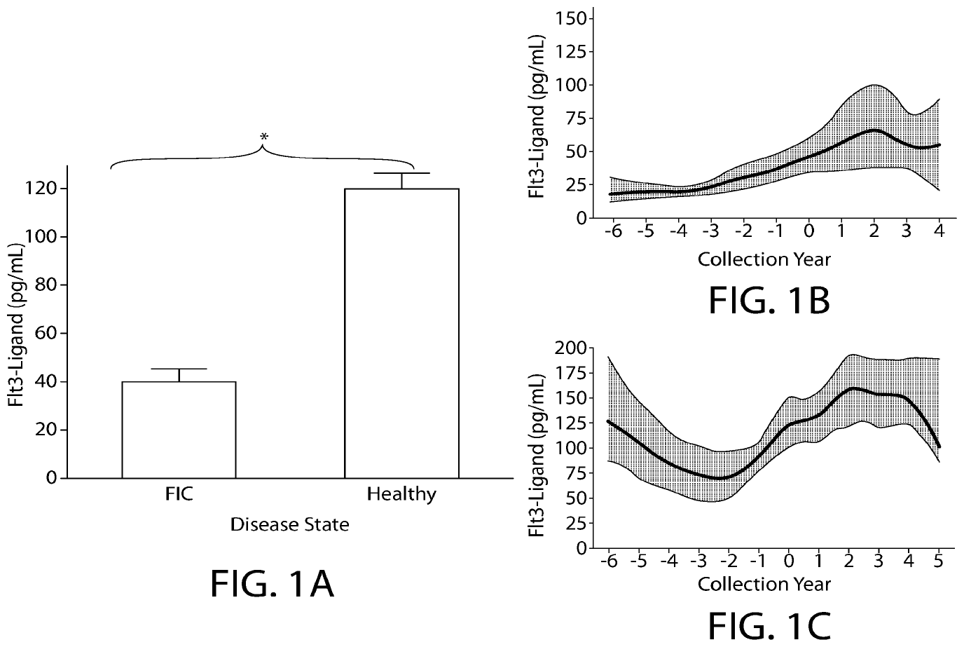

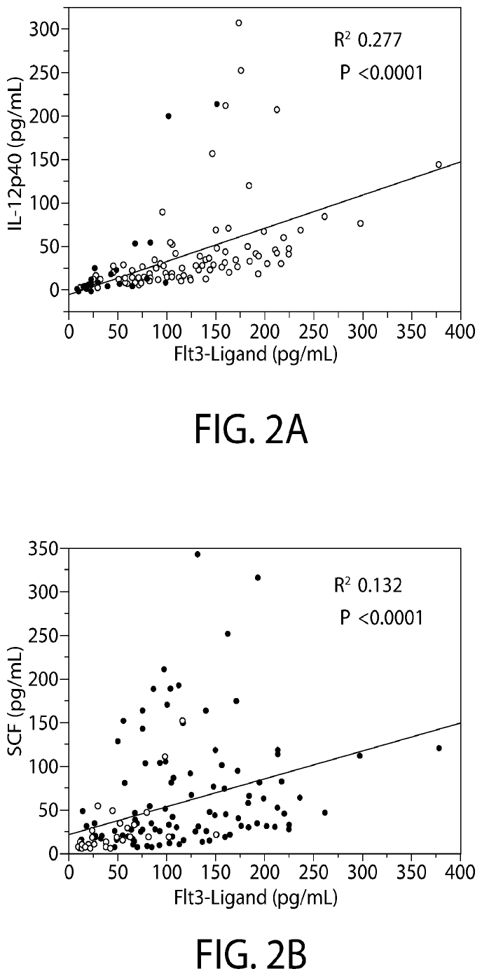

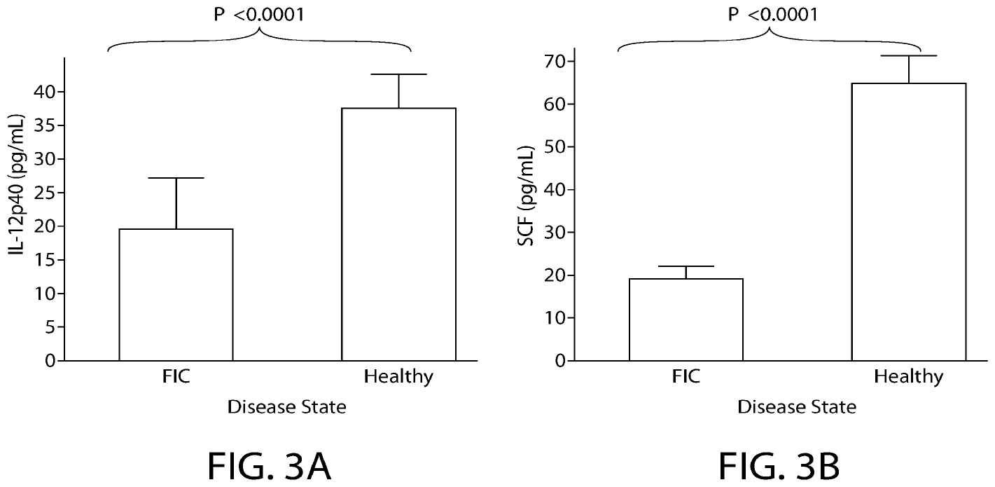

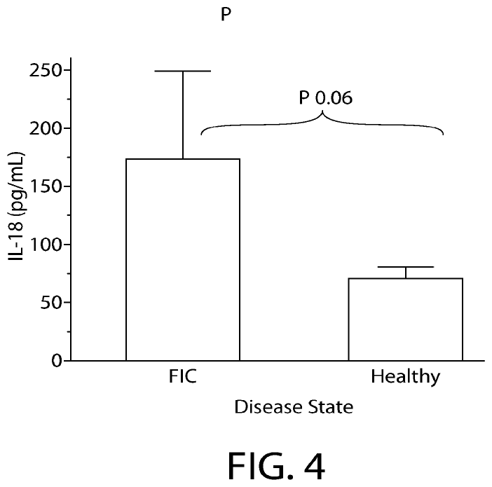

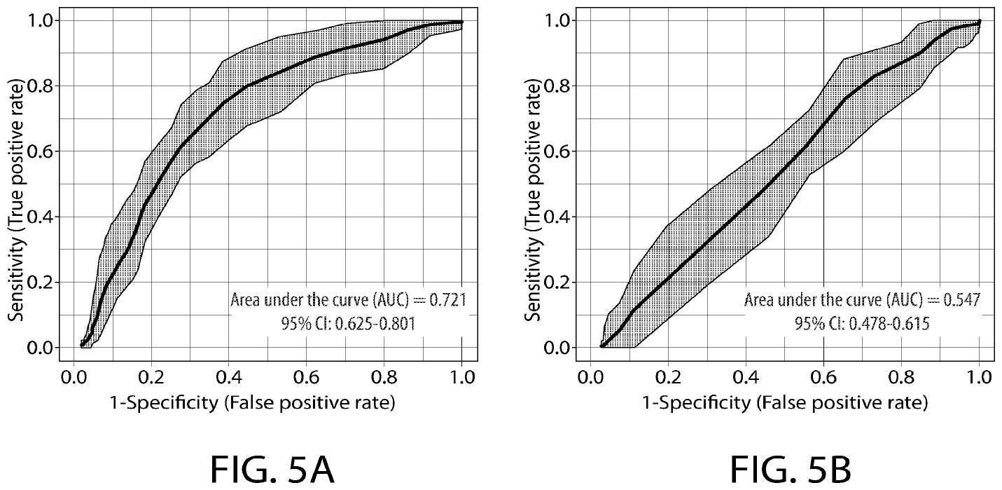

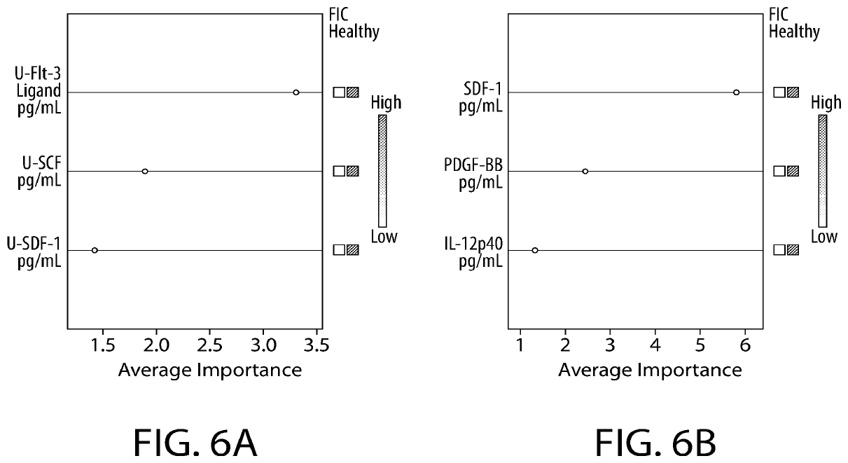

### DETAILED DESCRIPTION

The following invention relates in part to diagnostic methodology for assessing the propensity of felines to develop interstitial cystitis.

The following description of the preferred embodiment(s) is merely exemplary in nature and is in no way intended to limit the invention. The description of illustrative embodiments is intended to be read in connection with the accompanying drawings, which are to be considered part of the entire written description. The following description of the preferred embodiment(s) is merely exemplary in nature and is in no way intended to limit the invention. The description of illustrative embodiments is intended to be read in connection with the accompanying drawings, which are to be considered part of the entire written description.

As used herein and in the appended claims, the singular forms “a”, “an”, and “the” include plural references unless the context dictates otherwise. The singular form of any class of the ingredients refers not only to one chemical species within that class, but also to a mixture of those chemical species; for example, the term “protein” in the singular form, may refer to a mixture of compounds each of which is also considered a protein. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise defined, all technical and scientific terms and associated acronyms used herein have the same meaning as commonly understood by one of ordinary skill in the art. Further, it should be understood that the present invention is not limited to any particular method, protocol, and reagent described herein. The described methods, protocols, and reagents are merely examples and for illustration purposes only.

As used herein, the term “biological sample” may be used interchangeably with the terms “sample,” “specimen,” “biomaterial” and “biological material.” A biological sample refers to any organic material obtained from a pet including bodily fluids such as blood, saliva, and urine; tissue samples such as from a biopsy or fur; and other clinical specimens such as exhaled breath condensate. A biological sample could be obtained in a noninvasive and/or invasive manner. For example, a biological sample may be provided in such noninvasive ways as via a swabbing of a mouth, a collection of fur, or a urination. In other examples, a biological sample may be provided in such invasive ways as via a taking of blood via a needle or a removal of tissue via a biopsy.

In certain embodiments, the biological sample may further comprise one or more excipients. Excipients may be added to the biological sample at any time. For example, an excipient may be added to the biological sample during collection, transportation, preparation and/or analysis of the sample.

The addition of excipients are well known in the art. Such excipients should be present in amounts that do not impair the purpose and effect provided by the invention. An excipient may be included as a stabilizer, preservative, processing aid, pH buffer, bulking agent, diluent, color reagent and dye. For example, ethylenediaminetetraacetic acid (EDTA) may be added to a biological sample during collection to preserve the biological sample.

Examples of excipients may include boric acid and derivatives thereof, dimethyl sulfoxide (DMSO), ethanol, polyethylene glycol, ethylenediaminetetraacetic acid (EDTA), formic acid and derivatives thereof, protease inhibitors, sodium salts such as sodium citrate and sodium metabisulfate, and protease inhibitors.

A biosample (e.g., blood, saliva, urine, exhaled breath condensate, tissue, etc.) may be used as a diagnostic tool. For example, a biosample (such as a biofluid) may be used as a diagnostic tool due to its ability to correlate to a health status and/or condition of an animal (such as a person). The health condition may relate to a disease, disorder, or other condition. For example, a biofluid may be used to diagnose and/or determine an onset of a disease, progression of the disease, and/or treatment progress of the disease. A biofluid may be used as a diagnostic fluid in a noninvasive and/or invasive manner. For example, in some examples a biofluid may be provided in such noninvasive ways as via a swabbing of a mouth, a spitting of saliva from the mouth, or a urination. In other examples a biofluid may be provided in such invasive ways as via a taking of blood via a needle, a removal of tissue, etc. Detection (e.g., rapid detection) of antibodies in biofluids (such as saliva) may be performed rapidly and via compact tools (such as via a toothbrush, mouthguard, patch, etc.). The detection of a disease, disorder, or other condition via a biofluid may enable point-of-care diagnosis for diseases, disorders, or other conditions. Although the disclosure may provide many examples relating to biofluids, it should be understood that such examples are for illustration purposes only. The examples may extend to any biosample, including a biofluid as well as a biological tissue, for example.

The detection of a disease, disorder, or other condition via a biological sample may enable point-of-care diagnosis for diseases, disorders, or other conditions. Methods to quantify one or more biomarkers within a biological sample are well known in the art (e.g. colorimetric reporting, nuclear magnetic resonance (NMR) spectroscopy, infrared (IR) spectroscopy, mass spectrometry, etc.). Although the disclosure may provide many examples relating to urine, it should be understood that such examples are for illustration purposes only. The examples may extend to any biological sample, including a bodily fluid or biological tissue.

As used herein, the term “biomarker” may be used interchangeably with “biological marker” and is used to refer to any measurable substance that could be used to examine organ function or any other biological state or condition. In certain embodiments, a biomarker may include a protein such as an immunoglobulin, a polynucleotide such as DNA and RNA, and a metabolite. In certain embodiments, a biomarker may be a cytokine including, but not limited to, a colony stimulating factor (CSF), interferon (IFN), interleukin (IL), and tumor necrosis factor (TNF). In a preferred embodiment, the biomarker is an FMS-related tyrosine kinase 3 ligand biomarker. In another embodiment, the biomarker is a cytokine stem cell factor.

Detection and quantitation of biomarkers in a sample (such as urine) may be performed at any time after collection. For example, quantification of a biomarker within a biological sample may be performed within a short time period (e.g. within 2-minutes, within 5-mints, etc.) or after a longer storage, transportation, preservation, or incubation phase (e.g. within 6 hours, within, 72 hours, etc.). In certain embodiments, the biological sample is stored, transported, or preserved at temperatures ranging about from about −200° C. to about 30° C., e.g., −80° C. For another example, a tissue biopsy is preferably stored at −20° C. for short term storage and −80° C. for long term storage. It should be understood that these examples are for illustration purposes only and that the proper temperatures depend on the type of biological sample used in the present invention. The biological sample should be stored at the proper temperature for the specific type of biological sample as commonly understood by one of ordinary skill in the art.

One or more biomarkers within a sample may be used as a diagnostic tool for to any type of disease, disorder, or other condition. For example, one or more biomarkers may indicate a propensity (e.g., likelihood of developing) a cat may have for a disease, disorder, and/or condition. In a preferred embodiment, one or more biomarkers are used to diagnose or identify the propensity of interstitial cystitis in cats. In an even more preferred embodiment, FLT3L or cytokine SCF are used to diagnose or identify the propensity of interstitial cystitis in felines.

As described herein, an electronic assay may be used to quantify a biomarker in a biofluid (e.g., blood, saliva, urine, etc.). The biomarker may be associated with a disease, disorder, and/or condition. For example, a biomarker may indicate a presence of a disease, disorder, and/or condition. A biomarker may indicate a risk of (e.g., risk of developing) a disease, disorder, and/or condition. The electronic assay may use an impedance, such as impedance cytometry, to quantify the biomarker in a biofluid. A biomarker may include a protein. A biomarker may include an immunoglobulin G (IgG) and/or immunoglobulin A (IgA). Quantification of a biomarker within a biofluid may be performed within a short period of time (e.g., within 5 minutes, within two minutes, etc.) and via compact, lightweight, and/or inexpensive equipment. In embodiments, machine learning techniques (such as supervised machine learning techniques) may be used to determine (e.g., assist in determining) the quantification of biomarkers (such as immunoglobulins) within a biofluid.

In some embodiments, the present invention is a pet food composition used to treat any of the disease states or conditions described herein. In a preferred embodiment, the present invention is a method for treating interstitial cystitis in felines comprising administering a pet food composition. Preferably, the pet food composition comprises high levels antioxidants. In a more preferred embodiment, the pet food composition comprises high levels of antioxidants and is given daily for at least 28 days.

The pet food composition may be in the form of a kibble. In other embodiments, the pet food composition is in the form of multi-layer kibble and/or a multi-layer kibble comprising a coating. Further, the coating could comprise a palatant. The term “palatability”, as used herein, encompasses all the various properties of food sensed by animals such as texture, taste and aroma. For example, the coating may comprise a palatant to increase the palatability of the pet food composition. In certain embodiments, the composition has a palatability equal to that of a control composition.

In certain embodiments, the kibble is formed by extrusion. In other embodiments, the composition is in a form selected from: a loaf, a stew, a “meat and gravy” form, a gruel, shreds with a moisture content greater than 50% ”, and a product that could be pushed through a syringe. In another embodiment, the present invention comprises 6% wt. to about 12% wt. moisture.

In some embodiments, the kibble may comprise a binder. In certain embodiments the binder includes but is not limited to any of the following or combinations of the following: monosaccharides such as glucose, fructose, mannose, arabinose; di- and trisaccharides such as sucrose, lactose, maltose, trehalose, lactulose; corn and rice syrup solids; dextrins such as corn, wheat, rice and tapioca dextrins; maltodextrins; starches such as rice, wheat, corn, potato, tapioca starches, or these starches modified by chemical modification; alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, high lactose whey by-product, meat broth solids such as chicken broth, chicken broth solids, soy protein, and egg white.

In certain embodiments, the binder includes but is not limited to a lipid and/or lipid derivative. Lipids can be used in combination with water and/or other binder components. Lipids can include plant fats such as soybean oil, corn oil, rapeseed oil, olive oil, safflower oil, palm oil, coconut oil, palm kernel oil, and partially and fully hydrogenated derivatives thereof; animal fats and partially and fully hydrogenated derivatives thereof; and waxes.

In certain embodiments, the present invention may comprise additional ingredients including but not limited to, additives, minerals, vitamins, sources of carbohydrates, fat, protein, additional fiber, amino acids, carotenoids, antioxidants, fatty acids, glucose mimetics, probiotics, prebiotics, and others.

The pet food composition may contain additives known in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may increase the shelf life of the composition. Suitable examples can include preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches.

Additives for coloring, palatability, and nutritional purposes can include colorants, salts (including but not limited to sodium chloride, potassium citrate, potassium chloride, and other edible salts), vitamins, minerals, and flavoring. The amount of such additives in a composition typically is up to about 5% by weight (on a dry matter basis of the composition). In some instances, the pet food composition includes additives in an amount of up to about 4% by weight, up to about 3.5% by weight, up to about 3% by weight, up to about 2.5% by weight, up to about 2% by weight, up to about 1.5% by weight, up to about 1% by weight, based on the total weight of the pet food composition on a dry matter basis. Additionally or alternatively, the pet food composition may comprise about 1% by weight or less, about 0.5% by weight or less, or about 0.1% by weight or less of additive(s), based on the total weight of the pet food composition on a dry matter basis. Other additives can include antioxidants, omega-3 fatty acids, omega-6 fatty acids, glucosamine, chondroitin sulfate, vegetable extracts, herbal extracts, etc.

In certain embodiments, the pet food composition comprises vitamins and minerals in amounts required to avoid deficiency and maintain health. These amounts are readily available in the art. The Association of American Feed Control Officials (AAFCO) provides recommended amounts of such ingredients for dogs and cats (see Association of American Feed Control Officials. Official Publication, pp. 126-140 (2003)). Minerals may specifically be maintained at optimum levels known by those skilled in the art to reduce the incidence of stone formation.

Vitamins could as an example include vitamin A, vitamin B1 (thiamine or related sources such as thiamine mononitrate), vitamin B2 (riboflavin), vitamin B3 (niacin), vitamin B5 (pantothenic acid or related sources such as calcium pantothenate), vitamin B6 (pyridoxine or related sources such as pyridoxine hydrochloride), vitamin B8 (folic acid), vitamin B12, vitamin C (ascorbic acid), vitamin D (such as a vitamin D3 supplements), vitamin E, vitamin H (biotin), vitamin K, acetate, choline and choline related sources such as choline chloride, and inositol.

Minerals and trace elements could as an example include calcium, phosphorus, sodium, potassium, magnesium, copper, zinc, choline, and iron salts. Mineral sources can include, for example, sodium selenite, monosodium phosphate, calcium carbonate, potassium chloride, ferrous sulfate, zinc oxide, manganese sulfate, copper sulfate, manganous oxide, potassium iodide, and/or cobalt carbonate.

The term “carbohydrate” as used herein includes polysaccharides (e.g., starches and dextrins) and sugars (e.g., sucrose, lactose, maltose, glucose, and fructose) that are metabolized for energy when hydrolyzed. Examples of high carbohydrate ingredients suitable for inclusion in the compositions disclosed herein include but are not limited to, corn, grain sorghum, wheat, barley, and rice.

In certain embodiments, the carbohydrate component comprises a mixture of one or more carbohydrate sources. Examples of carbohydrate or carbohydrate ingredients may comprise cereals, grains, corn, wheat, rice, oats, corn grits, sorghum, grain sorghum/milo, wheat bran, oat bran, amaranth, Durum, and/or semolina.

One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like.

The carbohydrate mixture may additionally comprise optional components such as added salt, spices, seasonings, vitamins, minerals, flavorants, colorants, and the like. The amount of the optional additives is at least partially dependent on the nutritional requirements for different life stages of animals.

In some embodiments, the present invention may comprise about 5% wt. to about 25% wt. of fat. For example, the pet food composition may include fat in an amount from about 5% wt. to about 25% wt., about 5% wt. to about 20% wt., about 5% wt. to about 15% wt., about 5% wt. to about 10% wt.; about 10% wt. to about 25% wt., about 10% wt. to about 20% wt., about 10% wt. to about 15% wt.; about 15% wt. to about 25% wt., about 15% wt. to about 20% wt.; or about 20% wt. to about 25% wt., based on the total weight of the pet food composition. Sources of fats or fat ingredients may comprise poultry fat, chicken fat, turkey fat, pork fat, lard, tallow, beef fat, vegetable oils, corn oil, soy oil, cottonseed oil, palm oil, palm kernel oil, linseed oil, canola oil, rapeseed oil, fish oil, menhaden oil, anchovy oil, and/or olestra.

In some embodiments, the present invention may comprise about 5% wt. to about 30% wt. of protein. For example, the pet food composition may include protein in an amount from about 5% wt. to about 30% wt., about 5% wt. to about 25% wt., about 5% wt. to about 20% wt., about 5% wt. to about 15% wt., about 5% wt. to about 10% wt.; about 10% wt. to about 30% wt., about 10% wt. to about 25% wt., about 10% wt. to about 20% wt., about 10% wt. to about 15% wt.; about 15% wt. to about 30% wt., about 15% wt. to about 25% wt., about 15% wt. to about 20% wt.; about 20% wt. to about 30% wt., or about 20% wt. to about 25% wt., based on the total weight of the pet food composition. The term “protein” means a polypeptide, or a peptide, or a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompasses polymers of any length, including polymers containing from about 2 to 1000, from 4 to 800, from 6 to 600, and from 8 to 400 amino acids. The term includes amino acid polymers that are synthesized and that are isolated and purified from natural sources. Under some embodiments, the terms “polypeptide”, “peptide” or “protein” are used interchangeably.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthsize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

Examples of amino acids may comprise 1-Tryptophan, Taurine, Histidine, Carnosine, Alanine, Cysteine, Arginine, Methionine, Tryptophan, Lysine, Asparagine, Aspartic acid, Phenylalanine, Valine, Threonine, Isoleucine, Histidine, Leucine, Glycine, Glutamine, Taurine, Tyrosine, Homocysteine, Ornithine, Citruline, Glutamic acid, Proline, and/or Serine. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). In a preferred embodiment, the pet food composition contains high levels of arginine and derivatives thereof and/or low levels of tryptophan and derivatives thereof. In another preferred embodiment, the pet food composition contains high levels of polyunsaturated fatty acids (e.g., alpha linolenic, arachidonic, EPA and DHA)

Examples of fatty acid ingredients may comprise arachidonic acid, alpha-linolenic acid, gamma linolenic acid, linoleic acid, eicosapentanoic acid (EPA), docosahexanoic acid (DHA), and/or fish oils as a source of EPA and/or DHA. Sources of glucose mimetics may comprise glucose anti-metabolites including 2-deoxy Dglucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose.

Still other ingredients may include beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Larnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, and/or keratolytics.

The probiotic component may comprise any suitable bacteria, yeast, microorganisms, and/or mixtures of any thereof. Various probiotic microorganisms are known in the art. In certain embodiments, the probiotic component may comprise bacteria of the order Lactobacillus; bacteria of the genus Bacillus, Bacteroides, and/or Bifidobacterium; yeast of the order Saccharomyces including the genus Saccharomyces and Candida; and/or mixtures of any thereof. The probiotic may or may not form a spore.

In certain embodiments, the pet food composition may include polyphenols. In some embodiments, the polyphenol source comprises a phenolic compound selected from ellagic acid; gallic acid; protocatechuic acid; p-hydroxybenzoic acid; catechin; and a combination of two or more thereof. In some embodiments, the polyphenol source comprises pecan shells, or any other component of the pecan nut. Examples of further sources of polyphenols may comprise tea extract, rosemary extract, rosemarinic acid, coffee extract, pecan shells, caffeic acid, turmeric extract, blueberry extract, grape extract, grapeseed extract, and/or soy extract.

The pet food compositions may preferably include an effective amount of fiber-bound polyphenol component. The amount of fiber-bound polyphenol in the pet food compositions may, in some cases, be from about 0.1 wt. % to about 60 wt. %. For example, in various embodiments, the pet food composition may comprise fiber-bound polyphenol component in an amount from about 0.1 wt. % to about 60 wt. %, about 0.1 wt. % to about 50 wt. %, about 0.1 wt. % to about 40 wt. %, about 0.1 wt. % to about 30 wt. %, about 0.1 wt. % to about 25 wt. %, about 0.1 wt. % to about 20 wt. %, about 0.1 wt. % to about 17 wt. %, about 0.1 wt. % to about 14 wt. %, about 0.1 wt. % to about 11 wt. %, about 0.1 wt. % to about 9 wt. %, about 0.1 wt. % to about 7 wt. %, about 0.1 wt. % to about 5 wt. %, about 0.1 wt. % to about 3 wt. %; about 1 wt. % to about 60 wt. %, about 1 wt. % to about 50 wt. %, about 1 wt. % to about 40 wt. %, about 1 wt. % to about 30 wt. %, about 1 wt. % to about 25 wt. %, about 1 wt. % to about 20 wt. %, about 1 wt. % to about 17 wt. %, about 1 wt. % to about 14 wt. %, about 1 wt. % to about 11 wt. %, about 1 wt. % to about 9 wt. %, about 1 wt. % to about 7 wt. %, about 1 wt. % to about 5 wt. %, about 1 wt. % to about 3 wt. %; about 5 wt. % to about 60 wt. %, about 5 wt. % to about 50 wt. %, about 5 wt. % to about 40 wt. %, about 5 wt. % to about 30 wt. %, about 5 wt. % to about 25 wt. %, about 5 wt. % to about 20 wt. %, about 5 wt. % to about 17 wt. %, about 5 wt. % to about 14 wt. %, about 5 wt. % to about 11 wt. %, about 5 wt. % to about 9 wt. %, about 5 wt. % to about 7 wt. %; about 10 wt. % to about 60 wt. %, about 10 wt. % to about 50 wt. %, about 10 wt. % to about 40 wt. %, about 10 wt. % to about 30 wt. %, about 10 wt. % to about 25 wt. %, about 10 wt. % to about 20 wt. %, about 10 wt. % to about 17 wt. %, about 10 wt. % to about 14 wt. %; about 15 wt. % to about 60 wt. %, about 15 wt. % to about 50 wt. %, about 15 wt. % to about 40 wt. %, about 15 wt. % to about 30 wt. %, about 15 wt. % to about 25 wt. %, about 15 wt. % to about 20 wt. %; about 20 wt. % to about 60 wt. %, about 20 wt. % to about 50 wt. %, about 20 wt. % to about 40 wt. %, about 20 wt. % to about 30 wt. %, about 20 wt. % to about 25 wt. %; about 30 wt. % to about 60 wt. %, about 30 wt. % to about 50 wt. %, about 30 wt. % to about 40 wt. %; about 40 wt. % to about 60 wt. %, or about 40 wt. % to about 50 wt. %, based on the total weight of the pet food composition.

The pet food composition may be determined by any of the variety of methods for feed analysis known by one skilled in the art. Feed analysis may be done to measure any of the nutritional content listed herein including moisture, protein, fiber, carbohydrate, energy, vitamin, mineral, energy, fat, and ash content.

Protein content may be measured and reported in any of the variety of methods known to one skilled in the art. Protein may be reported as crude protein (CP) to measure both true protein content and non-protein nitrogen. Crude protein content may be further differentiated between degradable intake protein (DIP), undegradable intake protein (UIP) and metabolizeable protein (MP). In certain embodiments, protein content may be differentiated to include heat damaged protein or insoluble crude protein (ICP), adjusted crude protein (ACP), and digestible protein (DP).

Fiber content may be measured and reported in any of the variety of methods known to one skilled in the art. Fiber content may be reported as total dietary fiber (TDF, a combination of soluble and insoluble fiber) crude fiber (CF), neutral detergent fiber (NDF), acid detergent fiber (ADF) and/or acid detergent lignin (ADL). Crude fiber is generally known to estimate the indigestible portion of plant material found in pet food compositions. ADF measures cellulose and lignin, components of plant cell walls. NDF measures the total material found in plant cell walls and includes hemicellulose in addition to the fiber content measured as ADF. ADL measures only the lignin portion of a plant cell wall.

Energy content may be measured and reported in any of the variety of methods known to one skilled in the art. Energy content may be reported as digestible energy (DE), metabolizable energy (ME), net energy (NE), total digestible nutrient (TDN), ether extract (EE), relative feed value (RFV), and relative forage quality (RFQ).

Embodiments of the present invention will now be further described by way of the following, non-limiting, example.

### EXAMPLE

A retrospective study was done on serum and urine samples from 44 cats. Of the 44 cats, 6 cats had a clinical diagnosis of FIC, 3 cats had a clinical diagnosis of feline lower urinary tract disease (FLUTD), 13 cats had a clinical diagnosis of urinary bladder inflammation (UB), and 23 cats were used as healthy controls.

A cytokine analysis was performed on the serum and urine samples which measured 19 cytokines (sFas, TNFα, IL-12p40, SCF, PDGF-BB, IL-13, IL-18. IL-6, IL-4, IL-2, GM-CSF, KC, RANTES, SDF-1, FLT-3L, IL-1β, IFNγ, MCP-1 and IL-8). The cytokine analysis demonstrated that urinary cytokine Flt3-L was significantly reduced in FIC cats when compared with healthy cats (Table 1 below). Cells with an asterisk (*) indicate a clinically significant difference (p<0.05) between the groups.

TABLE 1
Mixed Model Analysis of Urinary Cytokines

| | | | | | Mixed model analysis/Factorial | | |
|---|---|---|---|---|---|---|---|
| | | | | | | | Heath |
| | | | | | Health | Collection | condition * |
| | FIC | FLUTD | Healthy | UB | condition | year | Collection |
| Analyte | Mean | Mean | Mean | Mean | (P value) | (P value) | year (P value) |
| SDMA (ug/dL) | 15.4954 | 13.8704 | 15.42857 | 19.45313 | 0.826873228 | 0.60863489 | 0.475370708 |
| Fas (pg/mL) | 1 | 1 | 1 | 1.056316 | 1 | 1 | 1 |
| Flt-3 Ligand | 39.9284 | 51.5183 | 119.5725 | 117.6468 | 0.003891235* | 2.22E−05* | 0.005878357* |
| (pg/mL) | | | | | | | |
| GM-CSF (pg/mL) | 0.90684 | 0.95667 | 1.004951 | 1.022368 | 0.070067829 | 0.365007901 | 0.337196898 |
| IFN-g (pg/mL) | 7 | 7.07333 | 7.025243 | 7 | 0.517767566 | 0.909932785 | 0.909932785 |
| IL-12p40 (pg/mL) | 19.6939 | 19.7444 | 37.63262 | 41.08553 | 0.362649536 | 0.005307006* | 0.189848406 |
| IL-13 (pg/mL) | 1 | 1 | 1 | 1.262368 | 1 | 1 | 1 |
| IL-18 (pg/mL) | 10.0621 | 8 | 8.42835 | 9.607368 | 0.237856419 | 0.94501972 | 0.808788397 |
| IL-1b (pg/mL) | 3 | 3.18167 | 3.275922 | 4.593684 | 0.576596228 | 0.545345377 | 0.545345377 |
| IL-2 (pg/mL) | 1.02789 | 1.53611 | 1.154563 | 1.748947 | 0.374811312 | 0.568171973 | 0.753213344 |
| IL-4 (pg/mL) | 0.06 | 0.06 | 0.064466 | 0.999737 | 0.631170912 | 0.56932751 | 0.56932751 |
| IL-6 (pg/mL) | 0.01 | 0.49 | 0.342524 | 2.138947 | 0.384974117 | 0.212989161 | 0.212989161 |
| IL-8 (pg/mL) | 17.0487 | 30.6344 | 55.59282 | 159.795 | 0.344251899 | 0.297585896 | 0.291429898 |
| KC (pg/mL) | 0.1 | 0.115 | 0.101942 | 0.843947 | 0.62919915 | 0.538132551 | 0.538132551 |
| MCP-1 (pg/mL) | 22.1282 | 19 | 19.69883 | 20.40263 | 0.309224685 | 0.910822718 | 0.644160645 |
| PDGF-BB (pg/mL) | 22.0042 | 24.1267 | 23.63 | 34.41605 | 0.423127772 | 0.292554958 | 0.29493229 |
| RANTES (pg/mL) | 0.25316 | 0.81667 | 2.199029 | 1.024474 | 0.982168745 | 0.987676504 | 0.986131826 |
| SCF (pg/mL) | 19.1424 | 33.1744 | 65.17913 | 57.22474 | 0.02475588* | 0.063895316 | 0.338500912 |
| SDF-1 (pg/mL) | 603.494 | 976.112 | 791.0553 | 856.4897 | 0.093514485 | 0.599714797 | 0.753623046 |
| TNF-a (pg/mL) | 2 | 2.26111 | 2.098835 | 3.511579 | 0.48109562 | 0.295081992 | 0.295081992 |

A univariate analysis was performed which identified that urinary Flt3-L and cytokine SCF had a 76% and 72% predictability of diagnosis, respectively. (Table 2 below). Cells with an asterisk (*) indicate a clinically significant difference (p<0.5) between the groups.

TABLE 2
Univariate Analysis of Top Analytes

| Analyte | AUC | P value |
|---|---|---|
| U-Flt-3 Ligand pg/mL | 0.765398551 | 1.31752E−08* |
| U-SCF pg/mL | 0.724637681 | 0.000152066* |
| U-SDF-1 pg/mL | 0.579257246 | 0.355406822 |
| S-SDF-1 pg/mL | 0.564893892 | 0.051522135 |
| S-PDGF-BB pg/mL | 0.591485507 | 0.71643046 |
| S-IL-12p40 pg/mL | 0.574340062 | 0.189408689 |

After analysis, the 2 cats clinically diagnosed with FIC were given a diet of ‘Feline Metabolic AOX upgrade dry’ or ‘Feline k/d Chick Canned’ cat food for 28 days. Urine collection occurred at 28 days (when experiment was ended) and after a few years following diagnosis. Cytokine analysis demonstrated that the urinary Flt3-L levels of the FIC group were close to the mean level of urinary Flt3-L levels in the healthy cat level.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1-44. (canceled)

45. A method of reducing risk of developing or delaying the onset and/or severity of interstitial cystitis in a feline subject, the method comprising:
identifying the feline subject having an increased risk of developing interstitial cystitis, wherein identifying the feline subject having the increased risk comprises:
analyzing a biological sample obtained from a feline subject to determine the concentration of a urinary cytokine;
analyzing a biological sample from a healthy feline subject to determine the concentration of the urinary cytokine;
comparing the urinary cytokine concentration in the feline subject to the urinary cytokine concentration of the healthy feline subject, wherein the feline subject's urinary cytokine concentration being less than the healthy feline subject's urinary cytokine concentration indicates that the feline subject has an increased risk of developing interstitial cystitis; and
administering to the feline subject a composition comprising an effective amount of a fiber-bound polyphenol component.

46. The method according to claim 45, wherein the urinary cytokine is selected from one or more of Fms-like tyrosine kinase 3 (Flt3-L), stem cell factor (SCF); IL-12p40, or a combination thereof.

47. The method according to claim 46, wherein the urinary cytokine is Flt3-L.

48. The method according to claim 45, wherein the fiber-bound polyphenol component comprises one or more of oat fiber; psyllium husks; tomato pomace; flaxseed; beet pulp; carrot powder; brewers rice; or a combination thereof.

49. The method according to claim 45, wherein the fiber-bound polyphenol component comprises one or more of oat fiber; psyllium husks; brewers rice; or a combination thereof.

50. The method according to claim 45, wherein the fiber-bound polyphenol component comprises one or more of tomato pomace; flaxseed; beet pulp; carrot powder; or a combination thereof.

51. The method according to claim 45, wherein the composition comprising the effective amount of fiber-bound polyphenol component is administered to the feline subject for at least about 14 days.

52. A kit for identifying a companion animal having an increased risk for developing interstitial cystitis, comprising:
a vessel for collecting a biological sample;
a detection method selected from: ELISA, chromatographic analysis, aptamer-based quantitative assay, fluorescence tags or stains specific to the combination of two or more metabolites thereof; point of care testing devices with preloaded chromophores/antibodies specific to one or more of the urinary cytokines described herein; and
instructions for use.

53. The kit according to claim 52, wherein the detection method evaluates the presence of a urinary cytokine selected from: Fms-like tyrosine kinase 3 (Flt3-L), stem cell factor (SCF); and IL-12p40, in a test animal.

54. The kit according to claim 53, wherein the instructions for use instruct the clinician to compare the presence of the urinary cytokine in the test animal to the presence of the urinary cytokine in a reference animal.

55. The kit according to claim 54, wherein the reference animal is a healthy animal.

56. The kit according to claim 54, wherein when the level of the urinary cytokine detected in the test animal is less than a level of the urinary cytokine detected in the reference animal, the test animal has an increased likelihood of developing interstitial cystitis.

57. A pet food composition comprising an effective amount of a fiber-bound polyphenol component, for use in increasing the level of a urinary cytokine selected from: Fms-like tyrosine kinase 3 (Flt3-L), stem cell factor (SCF); and IL-12p40, in a companion animal.

58. The pet food composition according to claim 57, wherein the fiber-bound polyphenol component comprises one or more of oat fiber; psyllium husks; tomato pomace; flaxseed; beet pulp; carrot powder; brewers rice; or a combination thereof.

59. The pet food composition according to claim 57, wherein the fiber-bound polyphenol component comprises one or more of brewers rice; oat fiber; psyllium husks; or a combination thereof.

60. The pet food composition according to claim 57, wherein the fiber-bound polyphenol component comprises one or more of tomato pomace; flaxseed; beet pulp; carrot powder; or a combination thereof.

61. A method of treating, inhibiting or ameliorating a symptom associated with interstitial cystitis in a companion animal, the method comprising administering the pet food composition of claim 57 to the companion animal in need thereof.

62. The method according to claim 61, wherein the fiber-bound polyphenol component of the pet food composition comprises one or more of oat fiber; psyllium husks; tomato pomace; flaxseed; beet pulp; carrot powder; brewers rice; or a combination thereof.

63. The method according to claim 61, wherein the fiber-bound polyphenol component comprises one or more of brewers rice; oat fiber; psyllium husks; or a combination thereof.

64. The method according to claim 61, wherein the fiber-bound polyphenol component comprises one or more of tomato pomace; flaxseed; beet pulp; carrot powder; or a combination thereof.
